(19) United States
(12) Plant Patent Application Publication
(10) Pub. No.: US 2006/0253946 P1
(43) Pub. Date: Nov. 9, 2006

(54) Kiwi plant named '51-1785'

(76) Inventor: Russell George Lowe, Te Puke (NZ)

Correspondence Address: GREENLEE WINNER AND SULLIVAN P C, 4875 PEARL EAST CIRCLE, SUITE 200, BOULDER, CO, 80301, US

(21) Appl. No.: 11/122,613

(22) Filed: May 5, 2005

(51) Int. Cl. A01H 5/00

(52) U.S. Cl. PLT/156

(57) ABSTRACT

ABSTRACT A new and distinct kiwi plant of the species Actinidia chinensis Planch. is described. The variety results from a controlled pollination using a female A. chinensis selection ‘Jing Feng’ (also known as 79-3, and by the HortResearch accession code CK34_01) of unknown parentage, and a male A. chinensis selection CK40_02 of unknown parentage. Both parents (‘Jing Feng’ and CK40_02) are unpatented. The new variety is distinguished by its large globose-shaped fruit with a flat stylar end, golden coloured flesh and tangy sweet taste.

### GENUS AND SPECIES OF PLANT CLAIMED

Actinidia chinensis

### VARIETY DENOMINATION

Not yet known.

### BACKGROUND TO THE INVENTION

Kiwi plants in cultivation are mainly varieties of A. deliciosa, particularly ‘Hayward’ although some A. chinensis and A. arguta varieties are grown. A. deliciosa and A. chinensis are closely related and varieties of both types have large fruit (˜100 g) with hair on the skin. The main varieties in New Zealand are ‘Hayward’ (A. deliciosa) and ‘Hortl6A’ (A. chinensis). Fruit are usually cut and eaten with a spoon.

All Actinidia species are dioecious, so female varieties have to be interplanted with male pollinizers to ensure fruit production.

A. chinensis vines are deciduous and tend to grow vigorously in spring and summer when rapidly-growing shoots can intertwine and tangle if not managed. Vines do best in a mild warm-temperate climate without late spring or early autumn frosts. They produce consistently heavy crops when grown in well-drained fertile soils and given regular irrigation in dry spells.

A. chinensis flowers in spring (mid October-early December) in New Zealand. Harvest of A. chinensis fruit may occur between April and late-May in New Zealand depending on the selection and location of plantings.

### SUMMARY OF THE INVENTION

The present invention is a new and distinctive kiwifruit variety having a generally globose shaped fruit with short, soft, silky hair and a golden yellow flesh when ripe. This new variety is designated ‘51-1785’ and is derived from a controlled pollination using a female tetraploid A. chinensis selection ‘Jing Feng’ (also known as 79-3, and by the HortResearch accession code CK34_{—}01) of unknown parentage, and a male tetraploid A. chinensis selection CK40_{—}02 of unknown parentage.

The female parent was introduced to New Zealand as vegetative plant material from Jiangxi, China. The male parent was selected in New Zealand from seedling plants raised from an introduction of seed from China in 1989. Both parents are unpatented.

This new variety was created during the course of a planned plant-breeding program, which was initiated during 1987 in Auckland, New Zealand. The cross was made on 24 Nov. 1994 in Te Puke, New Zealand. Seeds were sown in the winter of 1995 and 64 seedlings from this cross were planted out in the field at HortResearch Te Puke in August 1996. The selection ‘51-1785’ first flowered in November 1997 and fruit was first assessed in May 1998. Four existing kiwifruit vines in a clonal selection trial plot at HortResearch Te Puke were grafted to selection ‘51-1785’ in 1998, using graftwood from the original seedling plant.

The new variety can be asexually reproduced as cuttings or by grafting or budding on to seedling or cutting-grown rootstocks of A. deliciosa or A. chinensis. Trial plantings of grafted plants established at_the Te Puke, Nelson and Kerikeri Research Centres in 1998 have shown that the unique combination of characters come true to form and are established and transmitted through succeeding asexual propagations.

‘51-1785’ flowers later than Hortl6A and requires specific tetraploid males to ensure adequate pollination.

### BRIEF DESCRIPTION OF THE DRAWINGS

FIG. 1 shows typical fruit of ‘51-1785’ on the vine

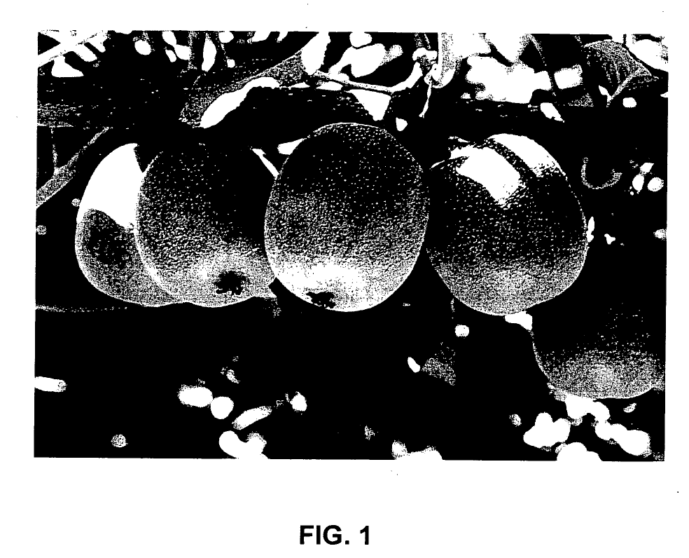

FIG. 2 shows typical fruit of ‘51-1785’ in side profile

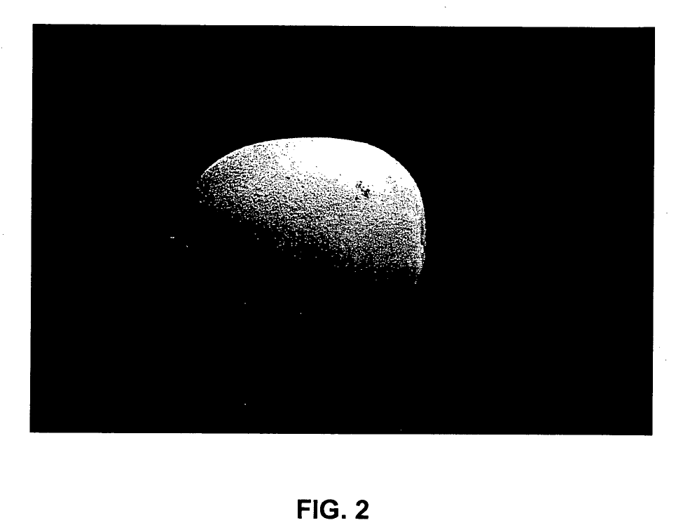

FIG. 3 shows a stem end view offruit of ‘51-1785’

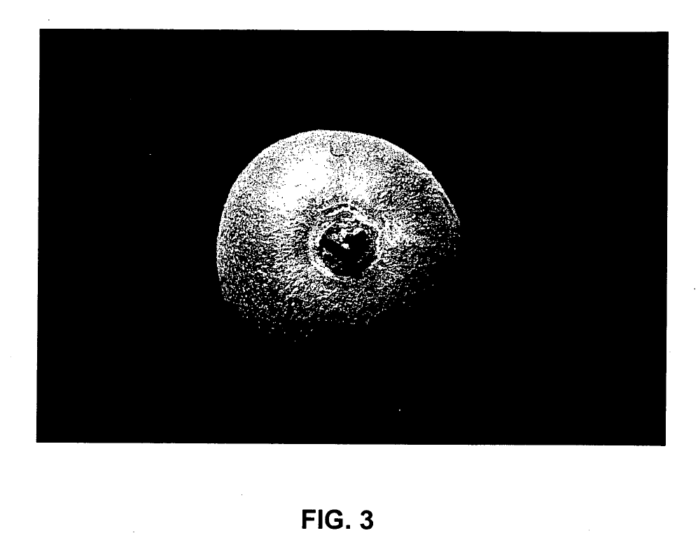

FIG. 4 shows a stylar end view of fruit of ‘51-1785’

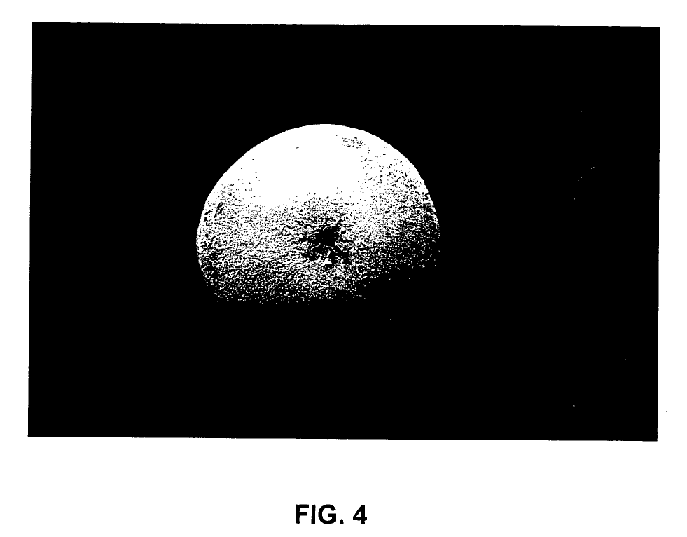

FIG. 5 shows fruit of ‘51-1785’ in cross section

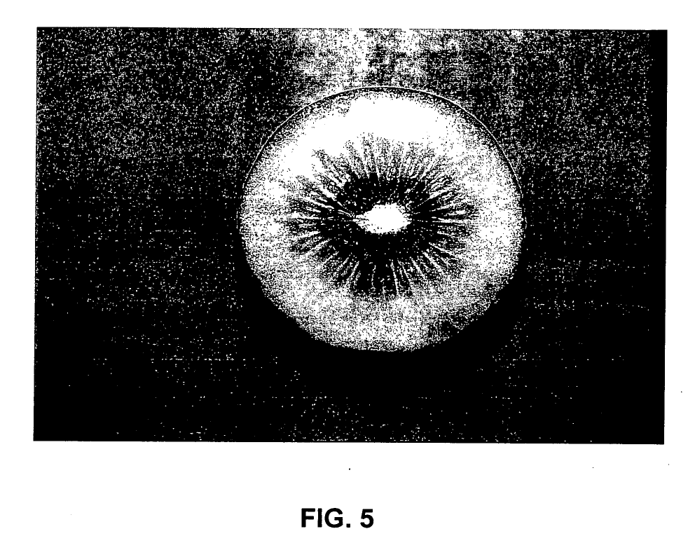

FIG. 6 shows flowers of ‘51-1785’

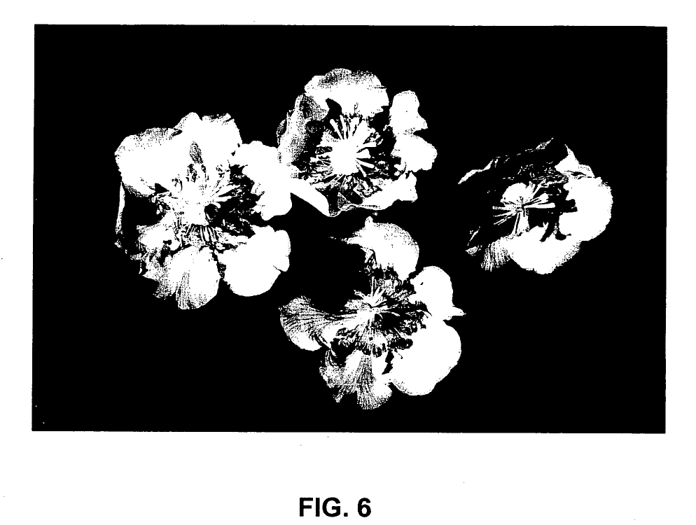

FIG. 7 shows the leaf of ‘51-1785’

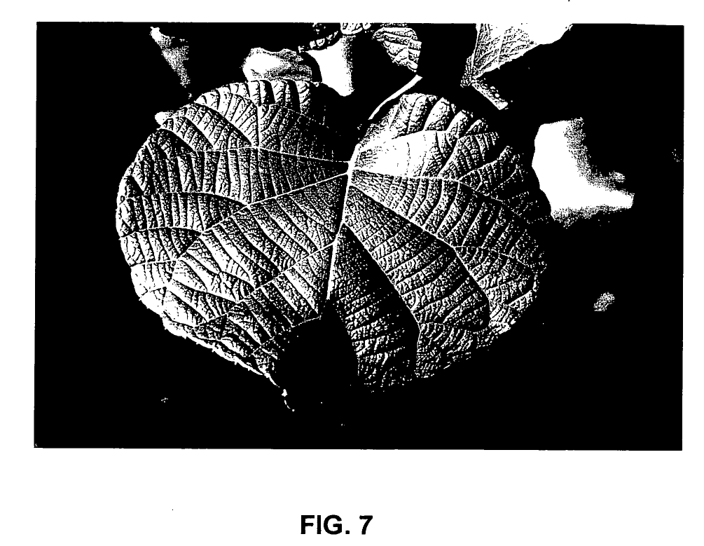

Photographs of fruit were taken at the normal harvest date. Fruit skin colour may vary depending upon extent of exposure to direct sunlight.

### DETAILED BOTANICAL DESCRIPTION

The new tetraploid kiwifruit variety ‘51-1785’ is pistillate (female), and produces imperfect flowers, i.e. the flowers produce only sterile pollen and require a pollinizer to set fruit.

TABLE OF CHARACTERISTICS
Horticultural terminology is used in accordance with revised
UPOV guidelines for kiwi. All dimensions in millimetres, weights
in grams (unless otherwise stated).

| | ‘51-1785’ |
|---|---|
| PLANT | |
| (Measurements from samples of 10, unless stated) | |
| Plant: sex expression | female |
| | flowers |
| | imperfect) |
| Plant: ploidy | tetraploid |
| | (2n = 2x = |
| | 116) |
| Plant: vigour | medium |
| Young shoot: hairs | present |
| Young shoot: density of hairs | medium |
| Young shoot: type of hairs | tomentose |
| Young shoot: anthocyanin coloration of growing tip | absent or |
| | very weak |
| Young shoot: anthocyanin coloration of leaf axil | absent or |
| | very weak |
| STEM | |
| Stem: coloration of leaf axil | absent or |
| | very weak |
| Stem: diameter | medium |
| Stem: dormant bud diameter | large |
| Stem: color on upper side of shoot | greyish- |
| | brown |
| Stem: character of bark | smooth |
| Stem: hairs | absent |
| Stem: conspicuousness of lenticels | conspicuous |
| Stem: number of lenticels | medium |
| Stem: color of lenticels | brown |
| Stem: size of bud support | medium |
| Stem: visibility of bud (dormant canes) | visible |
| Stem: number of hairs visible on bud (dormant canes) | many |
| LEAF (Mature) | |
| Leaf: general shape of blade | very broadly |
| | ovate |
| Leaf: length | 124 mm |
| Leaf: width | 151 mm |
| Leaf: petiole length | 103 mm |
| Leaf: shape of tip of blade | acute |
| Leaf: shape of base of blade | cordate |
| Leaf: arrangement of leaf bases | overlapping |
| Leaf: puckering/blistering on upper side of blade | medium |
| Leaf: margin | ciliate |
| Leaf: green color of upper side of blade | medium |
| Leaf: glossiness of upper surface of blade | medium |
| Leaf: color of lower side of blade | light green |
| Leaf: glaucosity (lower side of blade) | absent |
| Leaf: hairs on petiole | present |
| Leaf: density of hairs on petiole | medium |
| Leaf: anthocyanin coloration on upper side of petiole | medium |
| FLOWER | |
| Inflorescence: predominant number of flowers | three |
| Pedicel: hairs | present |
| Pedicel: length of hairs | very short |
| Flower: number of sepals | six or seven |
| Flower: color of sepals | green |
| Flower: diameter (terminal or king flower when fully open) | 51 mm |
| | (mean of 7 |
| | flowers) |
| Flower: mean number of petals per flower | 6 |
| Flower: arrangement of petals | overlapping |
| Flower: petal shoulder | present |
| Flower: primary color of petals (when fully open) | white |
| Flower: type of coloration of petals | bi-coloured |
| Flower: filament color | white |
| Flower: anther color | yellow |
| Flower: attitude of styles | semi-erect |
| Flower: curvature of styles | absent |
| Flower: amount of hair on ovary | dense |
| Flower: colour of ovary | white |
| FRUIT | |
| Fruit: overall size | 99 g |
| Fruit: length | 66 mm |
| Fruit: width (max) | 61 mm |
| Fruit: width (min) | 58 mm |
| Fruit: core diameter (max) | 12.4 mm |
| Fruit: core diameter (min) | 7.8 mm |
| Fruit: locule number | 30 |
| Fruit: peduncle length | 35 mm |
| Fruit: peduncle width | 3.9 mm |
| Fruit: general shape | globose |
| Fruit: cross section at median | circular |
| Fruit: general shape of stylar end | flat |
| Fruit: shape of shoulder on stalk end | squared |
| Fruit: skin color at harvest (fruit still hard) | medium |
| | brown |
| Fruit: skin colour change during ripening | absent |
| Fruit: skin color at maturity for consumption | medium |
| | brown |
| Fruit: hairs | present |
| Fruit: density of hairs | sparse |
| Fruit: type of hairs | pubescent |
| Fruit: hair length | short |
| Fruit: concentration of hairs | uniform |
| Fruit: adherence of hairs to skin (when rubbed) | weak |
| Fruit: core diameter (at largest diameter) | small |
| Fruit: core shape (in cross section) | transverse |
| | elliptic |
| Fruit: core woody spike | weak |
| Fruit: prominence of core woody spike | small |
| Fruit: outer pericarp color at maturity for consumption | yellow |
| Fruit: inner pericarp col. (locules) at mat. for consumption | yellow |
| Fruit: core color at maturity | yellow |
| Fruit: sweetness (Brix level) at maturity for consumption | 13.8% |
| | (11.4-16.2) |
| Fruit: vitamin C content (45 fruit sample) | 133 mg/100 |
| | g fresh |
| | weight (123- |
| | 140) |
| | mean of 5 |
| | plants, 3 val- |
| | ues, per |
| | plant. |
| Fruit: seed colour at maturity (in flesh) | blackish- |
| | brown |
| Fruit: seed colour when dry | dark brown |
| EVENTS | |
| Time of vegetative budbreak | medium |
| | (mid |
| | September) |
| Time of beginning of flowering | 8 Novem- |
| | ber (2004) |
| Time of maturity for harvest (at nominated Brix level) | last week of |
| | May (Brix |
| | 10%) |

Observations were made on plants growing at HortResearch Te Puke, Te Puke, New Zealand. These plants had been grafted on to seedling rootstocks.
Rootstocks:

‘51-1785’ vines can be grown on the same rootstocks as ‘Hortl6A’. Rootstocks currently being used in New Zealand include A. deliciosa and A. chinensis seedlings, ‘Hayward’ and ‘Kaimai’ rooted cuttings.
Cropping:

Young vines of ‘51-1785’ crop heavily when young, and must be thinned directly after fruit set to reduce crop loads and to ensure fruit size is not compromised by over cropping. Vines of ‘51-1785’ begin to bear fruit in their second year from graft and can be expected to reach full capacity at about 5 years. Storage Life

The storage life of ‘51-1785’ fruit is 20 weeks at 0C, if stored in unventilated containers.
Other data:

Fruit size.—Data gathered from harvesting all fruit, from 6 vines, in May 2004.

| | | | | | |
|---|---|---|---|---|---|
| Mean fruit weight: | 99.2 g | maximum: | 138 g | minimum: | 73 g |
| Mean fruit number: | 332 | maximum: | 1084 | minimum: | 102 |
| Mean yield: | 38.8 kg | maximum: | 14.1 kg | minimum: | 81.4 kg |

Fruit flesh and skin color measurement:

Color chart.—RHS Colour Chart, the Royal Horticultural Society, London, 1966.

| | |
|---|---|
| Fruit: core color at harvest | 150C to 150D |
| Fruit: seed color (in flesh) | 200A |
| Fruit: seed colour (dry seed) | 200C |
| Fruit: skin color at maturity | 199B |
| Leaf: color of upper side (in mature leaf after petal fall) | 147A |
| Leaf: color of lower side (in mature leaf after petal fall) | 148B |
| Plant stem: color on exposed side | 177A to 199A |

Comparison to closet cultivar:

The distinctive characteristics of this new kiwifruit variety, described in detail below, were observed in 2005 at HortResearch Te Puke, New Zealand. The age of the plants was 6 years from grafting onto seedling rootstocks.

Comparison with similar varieties ‘Hortl6A’ (U.S. Plant Pat. No. 11,066) and ‘KI 89’ (U.S. Plant Pat. No. 8,497) shows that ‘51-1785’ may be distinguished as follows in Table 1.

TABLE 1
Comparison with similar varieties

| Characteristic | ‘51-1785’ | Hort16A | KI 89 |
|---|---|---|---|
| Time of full bloom | late November | mid October | early |
| | | | November |
| Colour of fruit skin | mid-brown | yellow-brown | reddish- |
| | | | brown |
| Core diameter of fruit | very small | small | medium |
| General fruit shape | globose | ovoid | cylindrical |
| Fruit shape at stylar end | rounded | protruding | slightly |
| | | | depressed |
| Flesh color | golden yellow | yellow | yellow- |
| | | | green |
| Presence of lenticels on | present | not present | not |
| fruit skin | | | present |
| Visibility of lenticels on | conspicuous | not obvious | not |
| fruit skin | | | obvious |
| Mean soluble solids content | 12.5-14% | 14-19% | 10.8- |
| of ripe fruit | | | 12.4% |
| Mean dry matter at harvest | 17.8% | 18.3% | 15.5- |
| | | | 17.2% |
| Mean firmness at harvest | 6.8 Kgf | 4.6 Kgf | 3.7 Kgf |

Fruit of ‘51-1785’ is globose in shape with a rounded stylar end compared with the prominent stylar beak of ‘Hortl6A’. The flesh of ‘51-1785’ is golden yellow when ripe compared to the lighter yellow flesh of ‘Hort16A’ and the yellow-green flesh of ‘KI 89’ . The skin of ‘51-1785’ is a pale mid-brown colour, whereas the skin of ‘Hortl6A’ is yellow-brown and that of ‘KI 89’ is reddish-brown. The fruit of ‘51-1785’ has conspicuous, raised lenticels on the skin, ‘Hortl6A’ and ‘KI 89’ do not have raised lenticels.

The fruit of ‘51-1785’ is generally larger sized, has lower brix, lower dry matter content, and higher flesh firmness at harvest than Hortl6A. The fruit of ‘51-1785’ is generally shorter but of greater diameter than fruit of either ‘Hort16A’ or ‘KI 89’. claim:

1. A new and distinct kiwi plant of the species A. chinensis substantially as herein illustrated and described.
